(19) United States
(12) Patent Application Publication
(10) Pub. No.: US 2003/0223985 A1
(43) Pub. Date: Dec. 4, 2003

(54) Globulin protein to lower cholesterol in humans

(75) Inventors: Ralph D. Yoder, Ames, IA (US); Eric Matthew Weaver, Story City, IA (US)

Correspondence Address: MCKEE, VOORHEES & SEASE, P.L.C., 801 GRAND AVENUE, SUITE 3200, DES MOINES, IA, 50309-2721, US

(73) Assignee: The Lauridsen Group. Incorporated, Ames, IA (US)

(21) Appl. No.: 10/157,449

(22) Filed: May 29, 2002

(51) Int. Cl. A61K 39/395

(52) U.S. Cl. 424/130.1

(57) ABSTRACT

A globulin protein composition is described. The globulin concentrate is internally administered to animals including humans through food or water, or through conventional pharmaceutical dosage forms. The composition is effective in lowering blood cholesterol and phospholipids.

### BACKGROUND OF THE INVENTION

Cholesterol is found in practically all animal cells, serving as an essential component of the plasma membrane and other membrane structures. Cholesterol is a precursor of bile acids, the steroid hormones, and also vitamin D.

Cholesterol is not a dietary essential, since it is readily synthesized in the body. Dietary sources of cholesterol, amounting to 500 to 750 mg daily in the adult, are primarily animal products, meats, liver, eggs, and milk lipids, etc. Plant foods contain at most, only traces of cholesterol.

Once cholesterol is ingested, it is absorbed slowly from the gastrointestinal tract into the intestinal lymph. Food cholesterol is a variable mixture of free and esterified (with fatty acids) cholesterol. Esterified cholesterol is rapidly hydrolyzed in the intestine by a pancreatic esterase. Free cholesterol is incorporated into lipid micelles, together with conjugated bile acids and hydrolytic products of food fats. Absorption occurs mainly in the upper small intestine. On average, only about 50% of the cholesterol ingested in food and excreted in bile is absorbed.

After absorption, about 80-90% of the cholesterol is esterified by acylation with long-chain fatty acids in the presence of an esterase. The free and esterified cholesterol combine with specific apolipoproteins in the plasma to form chylomicrons and VLDL (very low density lipoproteins). These are then distributed to various tissue cells for metabolic disposal.

Besides the exogenous cholesterol absorbed each day from food entering the gastrointestinal tract, an even greater quantity of endogenous cholesterol is formed in the cells of the body. Essentially all the endogenous cholesterol that circulates in the lipoproteins of the plasma is formed by the liver, but all the other cells of the body form at least some cholesterol.

The formula of cholesterol is set forth below:

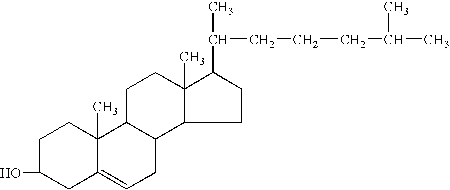

As illustrated, the basic structure of cholesterol is a sterol nucleus. This is synthesized entirely from multiple molecules of acetyl-CoA. In turn, the sterol nucleus can be modified by means of various side chains to form (a) cholesterol; (b) cholic acid; and (c) many important steroid hormones secreted by the adrenal cortex, the ovaries, and the testes.

By far the most abundant use of cholesterol in the body is to form cholic acid in the liver. As much as 80% of cholesterol is converted into cholic acid, which is then conjugated with other substances to form bile salts, which promote digestion and absorption of fats.

Cardiovascular disease (CVD) continues to be the leading cause of death in the U.S. Since numerous epidemiological studies have established that elevated total and LDL cholesterol are important risk factors for coronary artery disease (CAD), the use of cholesterol-lowering drugs has become a common medical practice. The cholesterol-lowering drug market is more than $10 billion and steadily increasing. In addition, individuals with mildly-elevated cholesterol also turn to foods and nutrients that are reported to have cholesterol lowering benefits and/or effects to improve their cardiovascular health. “Heart healthy” foods contribute many dollars in sales to the cardiovascular health category.

There are several factors that affect plasma cholesterol concentrations. One of these factors is an increase in the amount of cholesterol ingested each day. This, however, only increases the plasma concentration of cholesterol slightly, since the body has an intrinsic feedback mechanism to counteract the increase in exogenous cholesterol. Specifically, when cholesterol is ingested, the rising concentration of cholesterol inhibits one of the essential enzymes for endogenous synthesis of cholesterol.

Blood cholesterol levels are also influenced by both genetic and environmental factors. These include sex, age, diet, body-weight loss or gain, exercise, stress, and a number of pathological conditions. With increasing age animals experience a decreased capacity for fat metabolism. Cholesterol concentration in the tissues also increases. As an example, the amount of cholesterol in the aorta increases by approximately 1 mg of cholesterol per gram per decade.

Atherosclerosis is a disease of the intima of the arteries, especially of the large arteries, that leads to fatty lesions called atheromatous plaques on the inner surfaces of the arteries. The earliest stage in the development of these lesions is believed to be damage to the endothelial cells and sublying intima. The damage can be caused by physical abrasion of the endothelium, by abnormal substances in the blood, or even by the effect of the pulsating arterial pressure on the vessel wall. Once the damage has occurred, the endothelial cells swell and proliferate, and even sublying smooth muscle cells proliferate and migrate from the media of the arteries into the lesion. Soon thereafter lipid substances, especially cholesterol, begin to deposit from the blood in the proliferating cells, forming the atheromatous plaques. In the later stages of the lesions, fibroblasts infiltrate the degenerative areas and cause progressive sclerosis (fibrosis) of the arteries. Later, calcium often precipitates with the lipids to develop calcified plaques. When these processes have occurred, the arteries are then extremely hard, and the disease is called arteriosclerosis, or simply “hardening of the arteries”.

Arteriosclerotic arteries lose most of their distensibility, and because of the degenerative areas they are easily ruptured. Also, the atheromatous plaques of their surfaces causes blood clots to develop, with resultant thrombus or embolus formation. Almost half of all human beings in the United States and Europe die of arteriosclerosis. Approximately two thirds of these deaths are caused by thrombosis of one or more coronary arteries, and the remaining one third by thrombosis or hemorrhage of vessels in other organs of the body, such as in the brain which causes strokes, as well as in the kidneys, liver, gastrointestinal tract, and limbs.

An important part of this response to injury model in arteriosclerosis is the action of smooth muscle cells. Once the lumen of the vessel has been damaged by hypercholesterolemia, hypertension or some other pathological process, proliferation of smooth muscle cells occurs. This is followed by the formation of a connected tissue matrix, which comprises the groundwork for the atherosclerotic plaque. Smooth muscle cells are not only responsible for the formation of this matrix, but also contain the ability to express genes for a number of growth regulatory molecules, as well as receptors to growth factors. Thus, smooth muscle cells play a pivotal role in the pathogenesis of arteriosclerosis.

It has been known for some time that hypercholesterolemia is a risk factor in the development of arteriosclerosis. Treatments directed toward lowering blood cholesterol levels in individuals have included low-cholesterol diets, exercise, and treatment with nonprescription and prescription drugs. While these treatments have shown success in some individuals, others have been unable to benefit from these treatments.

For instance, many people are not able to or are unwilling to comply with low-cholesterol diets and exercise programs. Further, diet and exercise are often not sufficient in and of themselves in lowering highly elevated cholesterol levels.

With respect to pharmaceutical treatment of hyperlipidemia and/or hypercholesterolemia, conventional drugs have included niacin and other prescription antilipemic agents, such as lovastatin (Mevacor®) and gemfibrozil (Lopid®). These medications, however, are not well tolerated in some patients due to their associated side effects, which include flushing, abdominal pain, and liver function test elevations.

In recent years certain unique treatments for hyperlipidemia have been developed. For instance, it has been found that the administration of hyperimmune milk from bovines may be useful in reducing the accumulation of lipids, thereby preventing hypercholesterolemia, see Sharpe, “Cholesterol-Lowering and Blood Pressure Effects of Immune Milk”, Am. J. Clin. Nutr., 1994: 59:929-34. This general method is the subject matter of U.S. Pat. No. 4,636,384 to Stolle et al. The milk is only useful, however, if the milk-producing bovines are brought to a specific state of immunization by means of periodic booster administrations of an antigen or a mixture of antigens. The Sharpe reference suggests relative low dosage levels of globulin protein per day, i.e., 500 mg. of IgG/day.

It has recently been discovered that several animal species, including humans, have naturally-occurring antibodies to cholesterol. Alving, C. R. et al., Clin. Immunotherapeutics, 3:409 (1995). While it has been shown that not all animals have these antibodies, such as rabbits and mice, these animals readily gain them upon immunization with high-cholesterol liposomes containing lipid A or with intraperitoneal application of strong irritating adjuvants such as silicone oil that may cause recruitment of cholesterol as an antigen from cellular or necrotic debris to inflammatory sites. Alving, C. R. et al., Curr. Topics Microbiol. Immunol., 210:181 (1996). Researchers have developed a vaccine consisting of a protein-free liposome formulation loaded with cholesterol and lipid A as an antigen for inducing anti-cholesterol antibodies. Swartz, Jr., G. M., et al., Proc. Natl. Acad. Sci. USA, 85:1902 (1988). Studies in rabbits using these cholesterol liposomes resulted in reduced levels of diet-induced hypercholesterolemia. Alving, C. R., et al., J. Lab. Clin. Med., 127(1):40 (1996).

The present invention relates to the discovery that lipid and cholesterol absorption is significantly reduced if globulin protein is included in the diet of animals at certain defined levels, higher than taught by Sharpe, and it does not have to be derived from hyperimmune milk as in Sharpe.

It is an object of the present invention to provide a globulin protein supplement for treating animals which will decrease cholesterol and lipid absorption, thereby potentially lowering blood cholesterol levels.

Another object of the present invention is to provide a globulin protein supplement which is convenient and economical to administer, and which does not have to be derived from hyperimmune milk.

The method and means of accomplishing each of the above objectives as well as others will become apparent from the detailed description of the invention which follows hereafter.

### SUMMARY OF THE INVENTION

This invention provides for the first time a globulin protein supplement which is effective in lowering blood cholesterol and lipid absorption in animals including man. According to the present invention globulin protein consumed with cholesterol and other lipids significantly reduces the percentage and amount of cholesterol that is absorbed into the lymph system. This results in a significant reduction in absorbed blood cholesterol and lipids. Studies in animals show that the co-administration of plasma globulin protein and cholesterol results in a 38% reduction in cholesterol absorption. It also results in significant reduction of lipid for the first 5 hours post feeding, and the reduction in level is maintained even for weeks after administration stops.

### BRIEF DESCRIPTION OF THE DRAWINGS

FIG. 1 is a graph illustrating the amount of cholesterol absorbed over time when administered alone, with bovine serum albumin (BSA), and with globulin protein in accordance with the present invention. Squares represent values of cholesterol alone, circles represent cholesterol with BSA, and blackened circles represent cholesterol with globulin protein.

FIG. 2 is a graph illustrating the amount of phospholipid absorbed over time when administered alone, with BSA and with globulin protein. Squares represent values of the cholesterol alone, circles represent cholesterol with BSA, and blackened circles represent cholesterol with globulin protein.

FIG. 3 is a graph illustrating the lymph volume measured at hourly intervals following the administration of cholesterol alone (square), cholesterol with BSA (circles), and cholesterol with globulin protein (blackened circles). The subscripts indicate statistical significance between treatments.

FIG. 4 shows the effect of plasma derived IgG on total cholesterol.

FIG. 5 shows the effect of plasma derived IgG on glycerides.

FIG. 6 shows the effects of plasma derived IgG on LDL.

FIG. 7 shows the effects of plasma derived IgG on HDL.

FIG. 8 shows the effects of plasma derived IgG on the change in cholesterol:HDL ratio.

FIG. 9 shows the effects of plasma derived IgG on change in total cholesterol.

### DETAILED DESCRIPTION OF THE PREFERRED EMBODIMENT OF THE INVENTION

The present invention relates to a new method and composition for decreasing blood cholesterol and lipid absorption in animals through the administration of globulin protein. The invention also relates to a method of manufacturing said composition. The invention is applicable to any animal in which lower levels of cholesterol and lipid absorption are desired or necessary.

The present invention is in part predicated upon the discovery that the administration of globulin proteins from animal serum is effective in significantly reducing the percentage and amount of cholesterol absorbed into the lymph system. In addition, the phospholipid absorption is also reduced significantly. While sources have reported the existence of antibodies to cholesterol in animals, until now there have been no cholesterol-lowering studies conducted using the antibody-containing globulin proteins from animals.

Substantial evidence has been collected in animal studies that support an immunomodulating effect of orally-administered plasma proteins. Appetite, weight gain, lean mass, and measures of inflammation are all enhanced in immunocompromised animals given either immunoglobulin or plasma proteins orally. However, the inventors are not aware of any clinical studies that have reported changes in clinical chemistry or hematological measures in human subjects administered more than 1 g of native, natural bovine immunoglobulin.

As set forth above, there have been studies indicating that the administration of milk from cows challenged with enteric pathogens tends to reduce the level of blood cholesterol in humans. It has now been found that animal globulin proteins contain much higher levels of cholesterol antibodies than milk protein. Due to globulin protein's high concentration, animals can consume significant levels of the protein without significantly increasing their daily energy intake.

The present inventors unexpectedly discovered that globulin proteins, compared to other animal protein BSA, significantly decrease the absorption of cholesterol and other lipids. In fact, the inventor has found that BSA actually enhances the absorption of cholesterol and other lipids. And surprisingly, subjects actually maintained a lower cholesterol for several weeks after dosing stopped. While not wishing to be bound by a theory of operation, it is believed that the modulation of TNF may be involved in reducing circulating levels of cholesterol.

The composition of this invention is a plasma-based, substantially purified concentrate of globulin proteins. The globulin proteins may be administered as the serum isolated from whole blood. The serum source can be from any animal that has serum. In one embodiment of the invention the serum source is from the same species as that being treated. The serum is then administered to a human or animal, preferably orally. The serum product may be administered as a tablet, in food, or in water.

Using a globulin separation method, the globulin proteins may be further concentrated for administration as a globulin concentrate. The globulin concentrate is stable in water.

For purposes of human administration, the serum product or globulin concentrate may be administered through food, water, juices and other beverages, or milk products, such as yogurt. Such modes of administration are well known in the art, and have been used for such bacterial products as lactobacillus for many years.

The globulin concentrate can also be formulated into a pharmaceutical dosage form for oral administration, such as a tablet, capsule, suspension, granules, solution, etc. The pharmaceutical preparations of the present invention are manufactured in a manner which is itself well known in the art.

For example the pharmaceutical preparations may be made by means of conventional mixing, granulating, dragee-making, dissolving, lyophilizing processes. The processes to be used will depend ultimately on the physical properties of the active ingredient used. The term “pharmaceutically acceptable carrier” is herein defined as a non-toxic carrier that is compatible with the active and inactive ingredients of the formulations of this invention.

Suitable excipients are, in particular, fillers such as sugars for example, lactose or sucrose mannitol or sorbitol, cellulose preparations and/or calcium phosphates, for example, tricalcium phosphate or calcium hydrogen phosphate, as well as binders such as starch, paste, using, for example, maize starch, wheat starch, rice starch, potato starch, gelatin, gum tragacanth, methyl cellulose, hydroxypropylmethylcellulose, sodium carboxymethylcellulose, and/or polyvinyl pyrrolidone. If desired, disintegrating agents may be added, such as the above-mentioned starches as well as carboxymethyl starch, cross-linked polyvinyl pyrrolidone, agar, or alginic acid or a salt thereof, such as sodium alginate. Auxiliaries are flow-regulating agents and lubricants, for example, such as silica, talc, stearic acid or salts thereof, such as magnesium stearate or calcium stearate and/or polyethylene glycol. Dragee cores may be provided with suitable coatings which, if desired, may be resistant to gastric juices.

For this purpose concentrated sugar solutions may be used, which may optionally contain gum arabic, talc, polyvinylpyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethylcellulose phthalate, dyestuffs and pigments may be added to the tablet of dragee coatings, for example, for identification or in order to characterize different combination of compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, liquid paraffin, or liquid polyethylene glycols. In addition, stabilizers may be added. The serum or globulin protein fraction is placed in the carrier in a concentration that will deliver significantly more than 2 mg/kg bodyweight of immunoglobulin on a daily basis. For extreme hypercholesterolemia, higher doses may be necessary.

The globulin proteins of the present invention are derived from porcine or bovine serum, or other animal serum. The serum is obtained through conventional blood separation techniques. Typically, anticoagulant is first added to whole blood and then the blood is centrifuged to separate the plasma. Any anticoagulant may be used for this purpose, including sodium citrate and heparin. Persons skilled in the art can readily appreciate such anticoagulants. Calcium is then added to the plasma to promote clotting. This mixture is then centrifuged to remove the fibrin portion.

Once the fibrin is removed from plasma resulting in serum, the serum is used as a principal source of globulin protein. Alternatively, one could also inactivate this portion of the clotting mechanism through the addition of various anticoagulants.

In addition, one could simply administer the plasma into the carrier as the globulin protein. The serum is used as an immunoglobulin source in the globulin concentrate product. The serum may be further processed to increase the concentration of the protein component that blocks cholesterol and lipid absorption.

The final immunoglobulin concentrate can optionally be spray-dried into a powder. The powder allows for easier packaging and the product remains stable for a longer period of time than the raw globulin concentrate in liquid or frozen form. The immunoglobulin concentrate powder has been found to contain approximately 35-50% immunoglobulin proteins. The immunoglobulin concentrate is then ready for blending into user compounds for final application.

The immunoglobulin concentrate may then be prepared with one or more appropriate pharmaceutical excipients listed above into an oral dosage form for human or veterinary use.

Typical levels of immunoglobulin in serum from animals that naturally produce cholesterol antibodies appear to provide the basis for reducing cholesterol absorption. Hyperimmunization does not appear to be an essential component of the invention.

With respect to humans, the serum product can be administered through any of the aforementioned routes of administration in the same doses and concentrations cited above. For purposes of convenience, oral administration is preferred through such means as tablets or capsules, or as a supplement to milk products or water.

In general, the globulin proteins should be administered in a concentration of from about 15-30 mg immunoglobulin/kg bodyweight to decrease the absorption of cholesterol and other lipids. The preferred concentration is in the range of 25 to 30 mg globulin protein/kg bodyweight.

The globulin proteins may also be administered with certain additives or nutrients, such as carbohydrates, vitamins and minerals. The only requirement is that the additives also be compatible with immunoglobulin concentrate. Such additives can be readily ascertained by those skilled in the art.

The following examples are offered to illustrate but not limit the invention. Thus, they are presented with the understanding that various formulation modifications as well as method of delivery modifications may be made and still be within the spirit of the invention.

### EXAMPLE 1

### Effect of Globulin Protein Fraction on the Lymphatic Absorption of Cholesterol and Phospholipids in a Rat Model

The purpose of this example was to test the hypothesis that globulin protein lowers the intestinal absorption of exogenous cholesterol by its binding to luminal cholesterol and interfering with the incorporation of cholesterol into bile-salt micelles.

The experiment was conducted to evaluate the effect of globulin protein on cholesterol absorption in adult male rats without bile diversion (intact bile duct). This experiment consisted of the following 3 groups with 10 rats each:

1) infused with a lipid emulsion containing no globulin protein;

2) infused with a lipid emulsion containing a control protein (BSA); and

3) infused with a lipid emulsion containing globulin protein.

All experiments were conducted using male albino rats (Sprague Dawley, Harlan Sprague Dawley) weighing approximately 250 g. Upon their arrival, the rats were housed and acclimated for 2 weeks to a 12-hr light and dark cycle in a windowless room. All animals were cared for in the animal care facility of the Department of Foods and Nutrition, Kansas State University, according to the protocol approved by IACUC. Rats were fed a standard rodent diet formulated according to the American Institute of Nutrition 1993 (AIN-93) recommendations.

Rats were starved for 18 hr prior to surgery and anesthetized using a halothane vaporizer, which supplies halothane at the rate of 2% in 1.5 to 2.0 L oxygen/min. The major mesenteric lymph duct was cannulated using a soft vinyl tubing. An intraduodenal infusion catheter was placed by inserting silicone tubing (O.D., 2.1 mm) via the gastric fundus into the proximal duodenum.

The cannulation of the bile duct was performed as follows: Rats, starved for 18 hr, were anesthetized with halothane using a halothane vaporizer and a constant supply of oxygen as above. The abdomen was opened by a midline incision. The common bile duct was cannulated by inserting PE 10 tubing (Clay Adams, Parsippany, N.J.) and the bile was allowed to flow into a test tube upon insertion of the cannula. The cannula was secured in place with suture (4-0 silk, Ethicon Inc., Sommerville, N.J.) above the cannulation site and the common bile duct tied off below, with care taken not to tie off the entry of the pancreatic ducts. By passing the distal end of the cannula through a small incision on the side of the abdomen, the cannula was exteriorized. An intraduodenal infusion catheter was placed by inserting silicone tubing (O.D., 2.1 mm, Silastic, Dow-Corning, Michigan) via the gastric fundus into the proximal duodenum.

After closing the incisions, the rats were placed in restraining cages in a heated recovery chamber (30° C.) to prevent hypothermia and infused via the duodenal catheter with glucose saline (277 mM glucose, 144 mM NaCl, and 4 mM KCl) at 3 ml/h using an infusion pump (Harvard Apparatus, Model 935, South Natick, Mass.). The rat was allowed to recover for 20 hr (overnight).

After the post-operative period, the lymphatic absorption of cholesterol was measured by collecting the lymph at hourly intervals for 8 hr during infusion of a lipid emulsion at 3.0 ml/hr. The emulsion consisted of 500 mg triolein, 5 mg cholesterol labeled with 1.0 μCi of ¹⁴C-cholesterol, and a predetermined amount of globulin protein or BSA emulsified into 24 ml PBS (pH 6.4) by using Na-taurocholate. Lymph was collected in ice-chilled preweighed test tubes containing sodium EDTA and the hourly rate of lymph flow was measured. From hourly lymph samples, ¹⁴C radioactivity was determined in 100 μl aliquots after mixing with a scintillation fluid. The ¹⁴C radioactivity appearing in hourly lymph was expressed in dpm using an external standard method and quench corrections. From the radioactivity, the % dose of cholesterol absorbed at each hourly interval and for 8 hr was calculated.

Lipids were extracted from lymph, bile, and liver samples. Cholesterol and phospholipid was determined calorimetrically. Total cholesterol was separated into free and esterified cholesterol using digitonin.

The lymph ¹⁴C radioactivity was also separated into the free and esterified cholesterol fractions. The ¹⁴C radioactivity in each fraction was determined and the % distribution of ¹⁴C radioactivity was calculated.

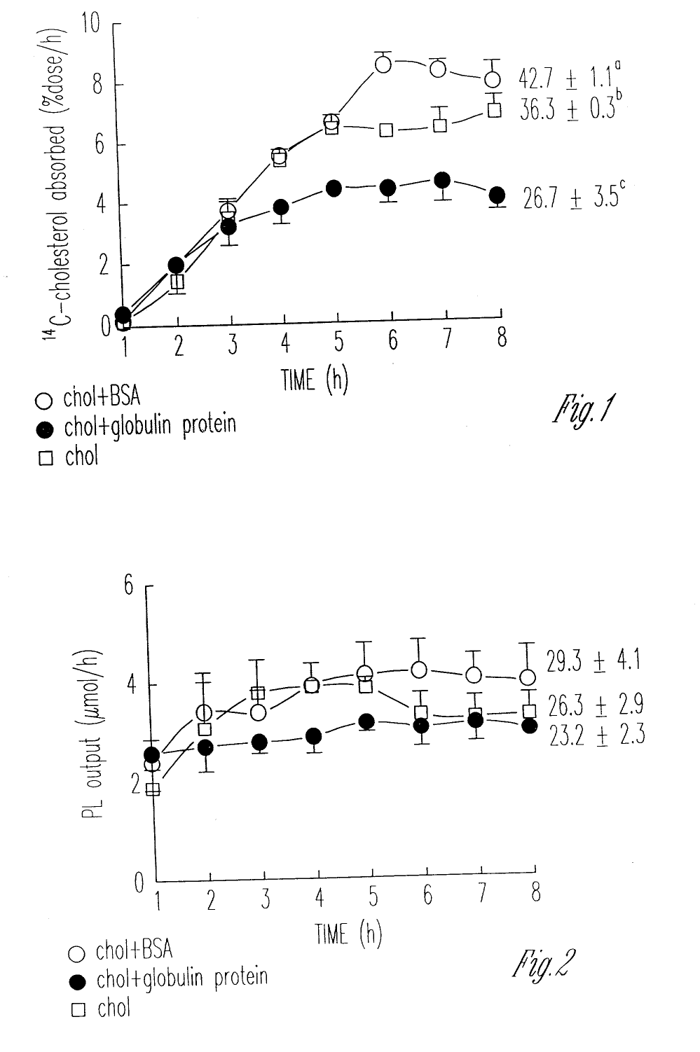

For the first 3 hours post feeding cholesterol absorption was the same for the 3 groups. After 3 hours the absorption rate per hour changed very dramatically. See FIG. 1. This clearly indicates that cholesterol absorption was reduced when globulin protein was included with the C-labeled cholesterol. These absorption values were significantly different between treatments. The magnitude of the difference at 6 hours through 8 hours post feeding is nearly a 50% reduction, and for the total period was a 38% reduction. These values had not previously been demonstrated and was not expected as result of this type of experiment. This is especially true when a comparable but different protein source, BSA, significantly enhanced the absorption of cholesterol. This measurement was also statistically significantly different from cholesterol alone.

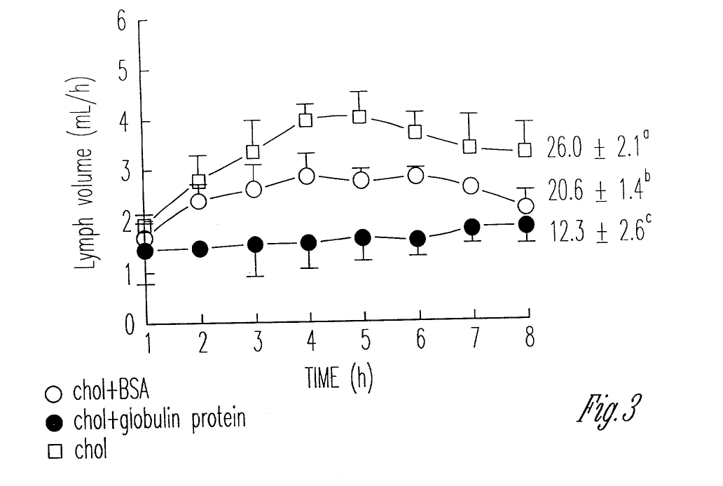

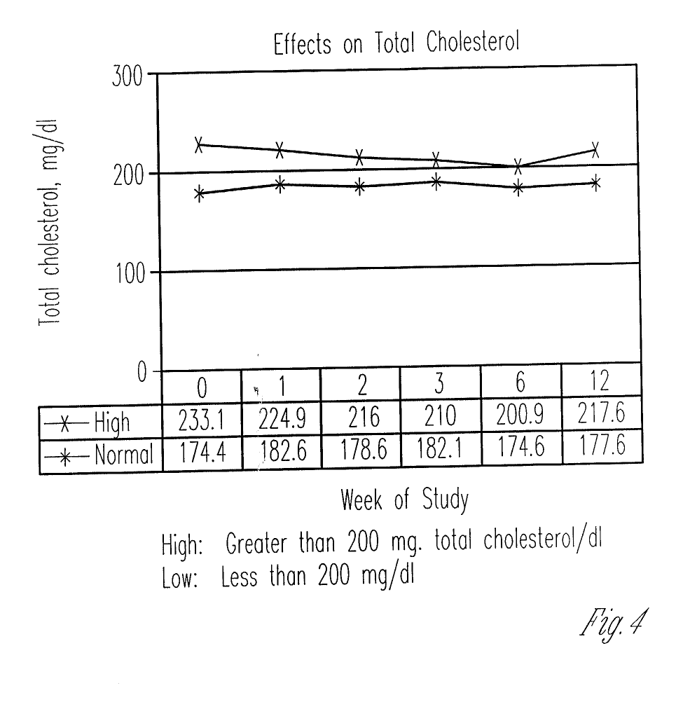

Phospholipid output was measured and is reported below in Table 1 and depicted in graph form in FIG. 2. In the globulin protein treatment the phospholipid output in the lymph was significantlly reduced for from hour 2 through hour 5 indicating that the globulin protein interferred with the absorption of phospholipids similar to that observed with cholesterol. This fact suggest that the actions of the globulin protein are general in nature and affect lipid absorption as well as cholesterol absorption.

TABLE 1
Hourly PL Output

| | Hour after intraduodenal infusion of lipid emulsion | | | | | | | | |
|---|---|---|---|---|---|---|---|---|---|
| Group | 1 | 2 | 3 | 4 | 5 | 6 | 7 | 8 | cumul. |

% dose/h

1. Group 1 (chol + albumin)

| | | | | | | | | | |
|---|---|---|---|---|---|---|---|---|---|
| X ± SD | 2.41^{a} ± | 3.42^{a} ± | 3.39^{ab} ± | 3.92^{a} ± | 4.10^{a} ± | 4.15^{a} ± | 4.01^{a} ± | 3.94^{a} ± | 29.33^{a} ± |
| | 0.47 | 0.79 | 0.45 | 0.43 | 0.62 | 0.62 | 0.47 | 0.68 | 4.11 |

2. Group 2 (chol + p-IgG)

| | | | | | | | | | |
|---|---|---|---|---|---|---|---|---|---|
| X ± SD | 2.61^{a} ± | 2.73^{a} ± | 2.81^{b} ± | 2.90^{b} ± | 3.16^{b} ± | 3.05^{b} ± | 3.14^{b} ± | 2.99^{b} ± | 23.20^{b} ± |
| | 0.76 | 0.51 | 0.23 | 0.34 | 0.18 | 0.38 | 0.38 | 0.10 | 2.29 |

3. Group 3 (chol)

| | | | | | | | | | |
|---|---|---|---|---|---|---|---|---|---|
| X ± SD | 1.89^{a} ± | 3.09^{a} ± | 3.78^{a} ± | 3.89^{a} ± | 3.85^{a} ± | 3.31^{b} ± | 3.24^{b} ± | 3.28^{b} ± | 26.34^{ab} ± |
| | 0.39 | 0.92 | 0.65 | 0.15 | 0.20 | 0.48 | 0.34 | 0.42 | 2.83 |

Cumulative PL output

| | Hour after intraduodenal infusion of lipid emulsion | | | | | | | |
|---|---|---|---|---|---|---|---|---|
| Group | 1 | 2 | 3 | 4 | 5 | 6 | 7 | 8 |

% dose

1. Group 1 (chol + albumin)

| | | | | | | | | |
|---|---|---|---|---|---|---|---|---|
| X ± SD | 2.41^{a} ± | 5.83^{a} ± | 9.21^{a} ± | 13.13^{a} ± | 17.23^{a} ± | 21.38^{a} ± | 25.39^{a} ± | 29.33^{a} ± |
| | 0.47 | 1.19 | 1.60 | 1.98 | 2.58 | 3.16 | 3.54 | 4.11 |

2. Group 2 (chol + p-IgG)

| | | | | | | | | |
|---|---|---|---|---|---|---|---|---|
| X ± SD | 2.61^{a} ± | 5.16^{a} ± | 7.96^{b} ± | 10.86^{a} ± | 14.02^{b} ± | 17.07^{b} ± | 20.21^{b} ± | 23.20^{b} ± |
| | 0.76 | 1.25 | 1.44 | 1.75 | 1.88 | 2.07 | 2.36 | 2.29 |

3. Group 3 (chol)

| | | | | | | | | |
|---|---|---|---|---|---|---|---|---|
| X ± SD | 1.89^{a} ± | 4.99^{a} ± | 8.77^{a} ± | 12.66^{a} ± | 16.51^{ab} ± | 19.81^{ab} ± | 23.06^{ab} ± | 26.34^{ab} ± |
| | 0.39 | 1.23 | 1.83 | 1.87 | 2.01 | 2.45 | 2.67 | 2.83 |

Means ± SD; n = 5 in group 1 & 2, n = 6 in group 3. Values in the same column not sharing a common superscript are significantly different (p < 0.05)

The lymph volume was also reduced by 40% when globulin protein was incorporated with cholesterol. See FIG. 2. In comparison, when BSA was incorporated with cholesterol the lymph volume went up 26% above the control and 111% above globulin plus cholesterol!

The above data clearly demonstrates that cholesterol and lipid absorption at the micelles level is affected when both globulin and BSA proteins are incorporated with cholesterol. These differences were completely unexpected.

### EXAMPLE 2 (HUMAN STUDY)

The objective of this study was to evaluate the effects of oral administration of immunoglobulin on clinical chemistry and hematological measures in healthy adult subjects that were mildly hypocholesterolemic.

The 12 week study was an uncontrolled, open-label study of the commercially-available dietary supplement ingredient ImmunoLin. The voluntary participants that took part in this study were informed of the product that was to be consumed as a part of the study and informed consent was obtained from all participants. All participants were made aware that they could discontinue his or her participation in the trial study at any time by notifying the study coordinator.

Twenty-two subjects volunteered for the study and all subjects completed the study. The immunoglobulin concentrate used for the study was prepared using FDA Good Manufacturing Practice guidelines for the production of foods for human consumption. The subjects were given two 2.5 g doses of the immunoglobulin concentrate per day, one in both the morning and afternoon, beginning on day 8 of the study. The product was pre-blended with 28.5 g of a cocoa-based beverage mix and then solubilized in approximately 250 ml of water prior to consumption.

The clinical chemistry measures evaluated in this study included: IgG, IgM, total cholesterol, triglycerides, calcium, blood urea nitrogen, creatinine, alkaline phosphatase, aspartate aminotransferase, and alanine aminotransferase. The hematological measures included: white blood cells, red blood cells, hemoglobin, hematocrit, platelets, mean corpuscular hemoglobin, mean corpuscular volume and mean corpuscular hematocrit.

The participants were asked to fast for 12-hours prior to collection of blood samples. Three blood samples were collected in both the first and second weeks of the study. Blood samples were collected once per week in weeks 3, 4, 6, and 12 of the study. Blood lipid analyses for total cholesterol, HDL cholesterol, and triglycerides were carried out by standard laboratory procedures. VLDL cholesterol and LDL cholesterol were calculated according to standard assumptions. VLDL was assumed to be 20W of triglycerides. LDL was calculated by subtracting HDL and VLDL from total cholesterol.

The data were analyzed using the Statistical Analysis System (SAS) software using Analysis of Variance (General Linear Models) procedures with a repeated measures approach. The means for weeks 2, 3, 4, 6, and 12 were all compared to week 1 (baseline). F-values were calculated and significance is reported at the 5% significance level.

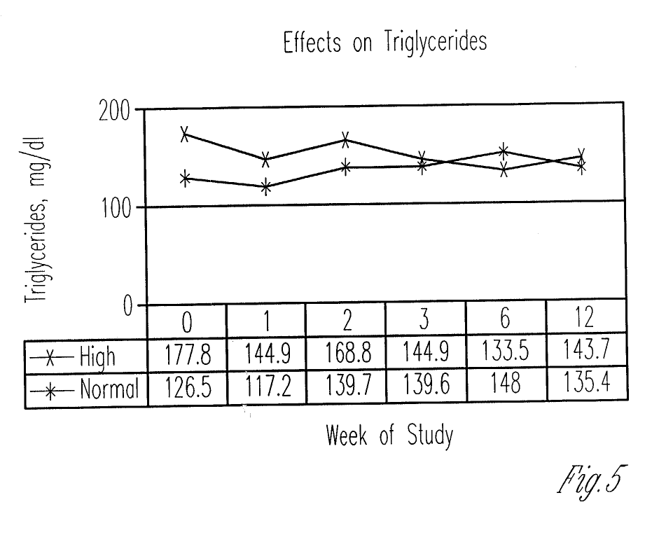

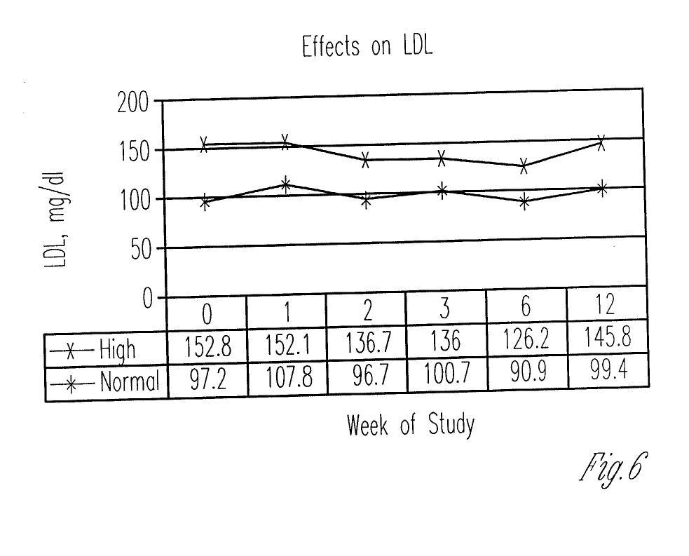

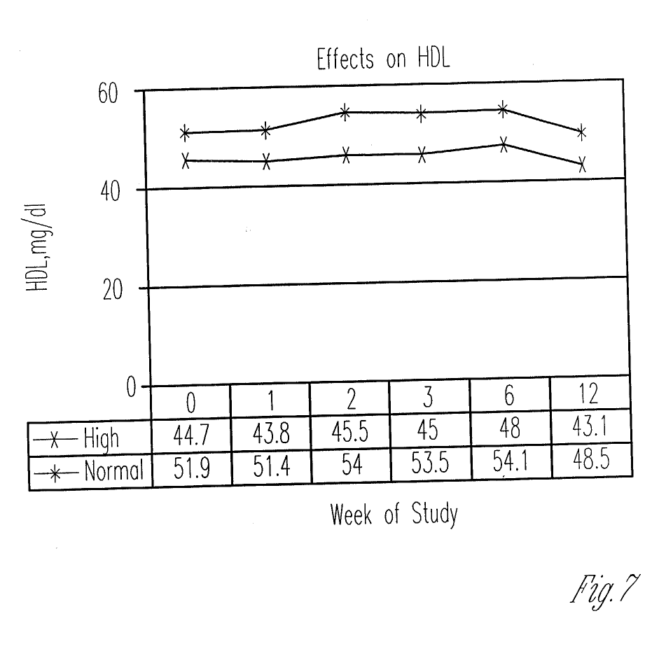

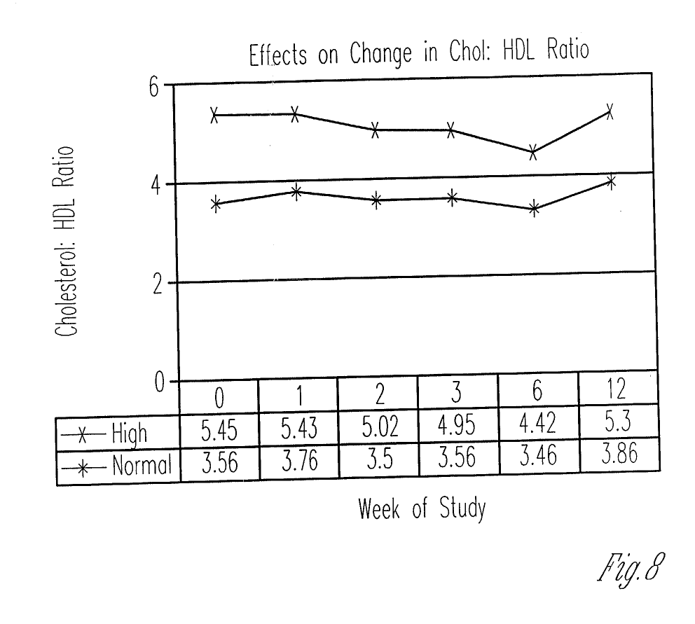

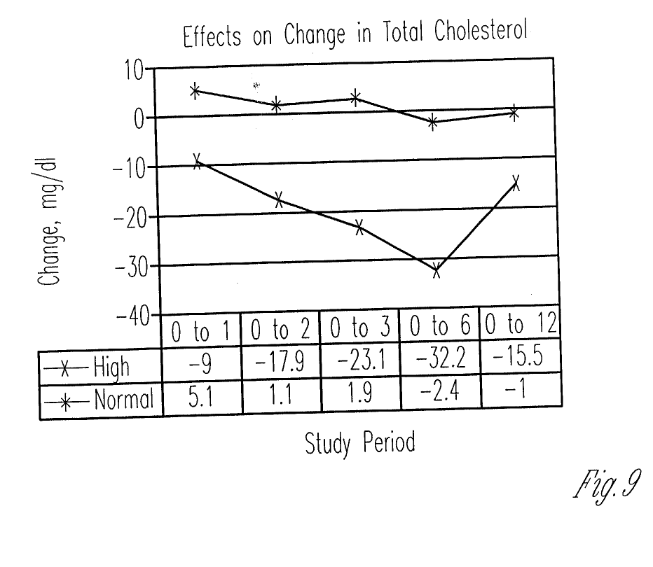

FIGS. 4-9 show data for the subjects at various designated intervals during the 12 week study. The data illustrated in these figures shows that the oral administration of bovine IgG concentrate reduced total and LDL cholesterol in healthy, mildly hypocholesterolemic volunteers. It further shows that immunoglobulin has an important immunomodulating role in the digestive tract. It is believed that TNF—a modulation helps explain the positive effects of plasma proteins on morbidity, appetite, and protein efficiency in immunocompromised animals, see co-pending and commonly assigned application of Campbell et al. entitled Methods and Composition for Treatment of Immune Dysfunction Disorders, Ser. No. 09/973,283, filed Oct. 9, 2001. While not wishing to be bound by any theory of operability, TNF-alpha modulation may also explain the effects of bovine IgG on mildly hypocholesterolemic volunteers.

From the above data in examples 1 and particularly example 2, it can be seen that surprising results are achieved without depending upon IgG from hyperimmune milk. Importantly, significantly lowered cholesterol effects are achieved and achieved for substantial periods of time after dosing ceases, providing that dosing is at a minimum acceptable level per day.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

What is claimed is:

1. A packaged composition for lowering blood cholesterol and lipids in animals including comprising:
packaged globulin proteins isolated from animal plasma and directions associated with the packaging for dosing at effective cholesterol lowering levels.

2. A composition according to claim 1 wherein the globulin concentrate contains at least 35% by weight IgG.

3. A composition according to claim 1 further including a pharmaceutically acceptable carrier.

4. A composition according to claim 1 that is formulated into a dosage form selected from the group consisting of tablet, capsule, solution, granules, powder, and suspension for internal administration.

5. A composition according to claim 1 which is processed to increase the total globulin content.

6. A composition according to claim 1 which is substantially purified.

7. A method of lowering blood cholesterol and lipid absorption in animals including humans comprising:
internally administering a composition to an animal in need of cholesterol lowering, said composition comprising globulin proteins isolated from animal plasma.

8. A method according to claim 7 wherein the source of animal plasma is bovine or porcine.

9. A method according to claim 7 wherein the composition is administered in a daily dose of from about 15 mg to about 30 mg of globulin protein per kilogram of bodyweight.

10. A method according to claim 7 wherein the composition is administered regularly with the ingestion of meals containing cholesterol and/or lipids.

11. A method according to claim 7 wherein the composition is administered through food or water.

12. A method according to claim 7 wherein the composition is administered in a dosage form selected from the group consisting of tablets, capsules, granules, liquid, suspension, and powder.

13. A method according to claim 7 wherein the composition is administered in food or drink selected from the group consisting of juice, milk, and dairy products.

14. A method according to claim 7 wherein the composition is administered orally.

15. A method of manufacturing a globulin protein composition for lowering blood cholesterol and lipid absorption in animals including humans comprising:
obtaining blood from an animal source, wherein the blood includes a plasma and a red cell fraction;
separating the plasma from the red cell fraction;
removing fibrin from the plasma to form serum;
removing a portion of the albumin to form globulin protein concentrate;
placing the globulin protein fraction in a pharmaceutically acceptable carrier; and
directing dosing at a level of from about 15 mg/kg bodyweight to about 30 mg/kg bodyweight for cholesterol reduction.

16. A method according to claim 15 wherein the globulin protein fraction is placed in the carrier in a concentration of from about 5% to about 95% by weight.

17. A method according to claim 15 wherein the globulin protein fraction is further processed to increase the concentration of immunoglobulins.

18. A method according to claim 15 further including the step of spray-drying the composition into a powder.
